(19) United States
(12) Patent Application Publication
(10) Pub. No.: US 2017/0293727 A1
(43) Pub. Date: Oct. 12, 2017

(54) INTELLIGENT BLOOD PRESSURE MONITORING

(71) Applicant: APPLE INC., CUPERTINO, CA (US)

(72) Inventors: Erno H. KLAASSEN, Los Altos, CA (US); Thomas J. SULLIVAN, San Jose, CA (US); Stuart L. GALLANT, Morgan Hill, CA (US); Michael O'REILLY, San Jose, CA (US); Zijing ZENG, San Jose, CA (US); Gracee AGRAWAL, Cupertino, CA (US)

(21) Appl. No.: 15/094,978

(22) Filed: Apr. 8, 2016

(51) Int. Cl. G06F 19/00; A61B 5/024; A61B 5/022; A61B 5/16; A61B 5/11; A61B 5/00

(52) U.S. Cl. CPC G06F 19/3406; A61B 5/1118; A61B 5/742; A61B 5/1112; A61B 5/022; A61B 5/6801; A61B 5/6898; A61B 5/7475; A61B 5/165; A61B 5/0022; A61B 5/024; A61B 5/486; A61B 2562/0219

(57) ABSTRACT

In some implementations, a user device can assist a user with intelligent blood pressure monitoring. For example, the user device can present notifications and/or reminders that prompt the user to take blood pressure measurements at a prescribed time or according to a prescribed schedule. The user device can automatically determine that the user should or should not take a blood pressure measurement based on the user's context and suggest an alternative time for taking the blood pressure measurement. For example, the user's context can include the user's physical and/or psychological state inferred based on sensor data, application data, and/or other detectable information. In some implementations, the user device can automatically monitor the user's blood pressure and take blood pressure measurements based on user context triggers.

### TECHNICAL FIELD

The disclosure generally relates to collecting biometric data.

### BACKGROUND

Patients are often required by their doctor to monitor heart rate, blood pressure, and other biometric data so that the doctor can detect or identify abnormalities or ailments before the patient suffers some irreversible damage. Typically, it is up to the patient to remember to take the patient's blood pressure according to a schedule prescribed by a doctor. Sometimes the patient will take blood pressure measurements according to the prescribed schedule but the patient's recent activity, environment, or other stimuli will result in an erroneous blood pressure measurement.

### SUMMARY

In some implementations, a user device can assist a user with intelligent blood pressure monitoring. For example, the user device can present notifications and/or reminders that prompt the user to take blood pressure measurements at a prescribed time or according to a prescribed schedule. The user device can automatically determine that the user should or should not take a blood pressure measurement based on the user's context and suggest an alternative time for taking the blood pressure measurement. For example, the user's context can include the user's physical and/or psychological state inferred based on sensor data, application data, and/or other detectable information. In some implementations, the user device can automatically monitor the user's blood pressure and take blood pressure measurements based on user context triggers.

Particular implementations provide at least the following advantages. The user device can help the user take more consistent blood pressure readings. The user device can provide context for the blood pressure readings so that a reviewing doctor can make more informed decisions about the user's health. The user device can identify suitable times for resting, active, and stressed blood pressure measurements so that the measurements are most representative of these physical states of the user.

Details of one or more implementations are set forth in the accompanying drawings and the description below. Other features, aspects, and potential advantages will be apparent from the description and drawings, and from the claims.

### DESCRIPTION OF DRAWINGS

FIG. 1 is a block diagram of an example system for intelligent blood pressure monitoring.

FIG. 2A and FIG. 2B are example graphs illustrating adapting a blood pressure measurement schedule based on user context.

FIG. 3 illustrates an example graphical user interface for receiving blood pressure measurement input from a user.

FIG. 4 illustrates an example graphical user interface for initiating a blood pressure measurement.

FIG. 5 illustrates an example graphical user interface for reminding the user to take a blood pressure measurement and to suggest that the user rest for a period of time before taking the blood pressure measurement.

FIG. 6 illustrates an example graphical user interface for prompting take a blood pressure measurement in advance of a predicted user stress period.

FIG. 7 illustrates an example blood pressure measurement database.

FIG. 8 illustrates an example graphical user interface for presenting a blood pressure measurement summary to the user.

FIG. 9 illustrates an example graphical user interface for describing the user context associated with a blood pressure measurement.

FIG. 10 illustrates an example graphical user interface for presenting a detailed view of a user's recorded blood pressure measurements.

FIG. 11 is flow diagram of an example process for intelligent blood pressure monitoring.

FIG. 12 is a flow diagram of an example process for automatic blood pressure monitoring.

FIG. 13 is a block diagram of an exemplary system architecture implementing the features and processes of FIGS. 1-12.

Like reference symbols in the various drawings indicate like elements.

### DETAILED DESCRIPTION

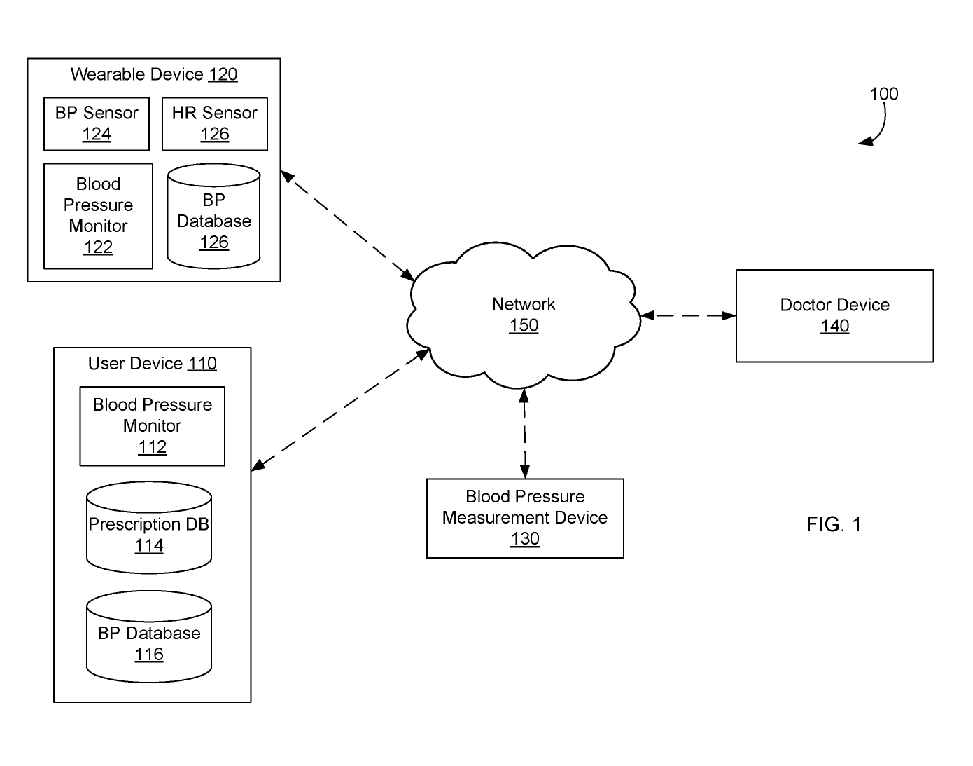

FIG. 1 is a block diagram of an example system 100 for intelligent blood pressure monitoring. For example, system 100 can determine the most suitable times for measuring a user's blood pressure and present notifications reminding the user to take a blood pressure measurement. In some implementations, the user can use a separate blood pressure measuring device and manually input the blood pressure measurements into a user device. In some implementations, the user device (e.g., a wearable device) can automatically take the user's blood pressure measurements.

In some implementations, system 100 can include user device 110. For example, user device 110 can be a desktop computer, a laptop computer, a handheld device, a smartphone, a tablet device, or any other computing device. User device 110 can be associated with a user (e.g., an owner, operator, etc.) of user device 110. The user of user device 110 can be a patient of a doctor associated with doctor device 140, for example.

In some implementations, the doctor can use doctor device 140 to write a prescription for the user. For example, the prescription can indicate that the user should take heart rate and/or blood pressure measurements according to a prescribed schedule (e.g., on a periodic time interval, at a specified time and day, etc.). After the doctor has generated the prescription, doctor device 140 can transmit the prescription to user device 110 through network 150. For example, network 150 can be a wired or wireless network. Network 150 can be a local area network (LAN), wide area network (WAN), and/or the Internet. Network 150 can be a peer-to-peer network that uses a near field communication technology, Bluetooth, Bluetooth low energy, Wi-Fi, or some other direct device to device networking technology.

In some implementations, user device 110 can store a prescription for the user. For example, user device 110 can receive a prescription for medication and/or blood pressure measurements from doctor device 140. Alternatively, the user of user device 110 can manually enter prescription information (e.g., using an graphical user interface, form, etc.) presented on a display of user device 110. After user device 110 receives the prescription (e.g., from the user, from doctor device 110, etc.), user device 110 can store the prescription in prescription database 114.

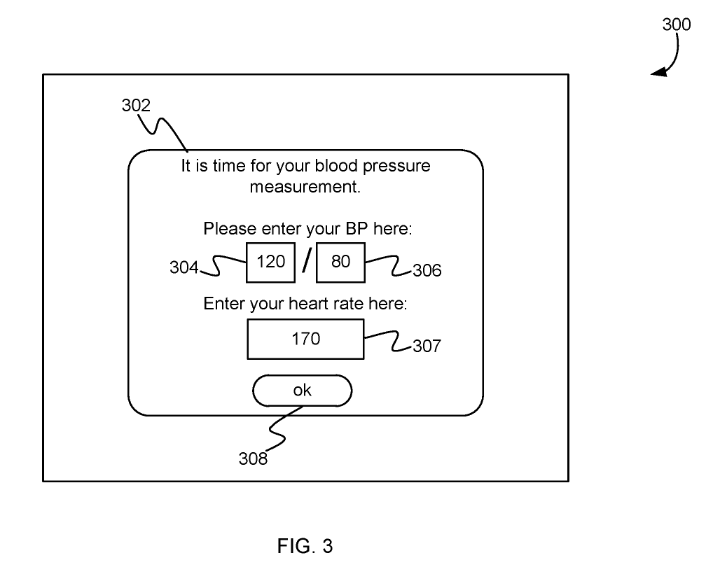

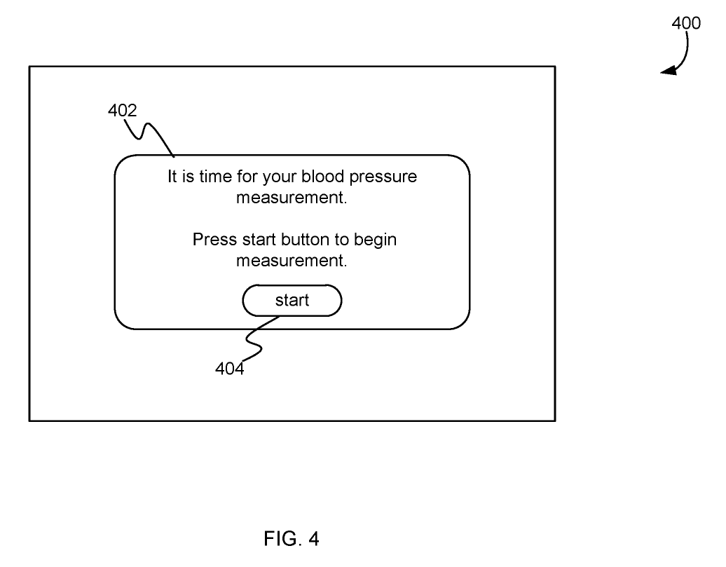

In some implementations, user device 110 can include blood pressure monitor 112. For example, when user device 110 receives a prescription (e.g., medication and/or blood pressure measurement), blood pressure monitor 112 can schedule notifications to remind the user to measure the user's blood pressure and/or take the prescribed medication according to the prescribed schedule. In some implementations, blood pressure monitor can present a notification reminding the user to measure the user's blood pressure at the prescribed time. For example, blood pressure monitor 112 can present a notification (e.g., notification 302 of FIG. 3, notification 402 of FIG. 4, etc.) every 4 hours reminding the user to take a blood pressure measurement. The notification can be presented graphically on a display of user device 110, audibly using speakers of user device 110, using haptic feedback (e.g., vibrations), or a combination thereof.

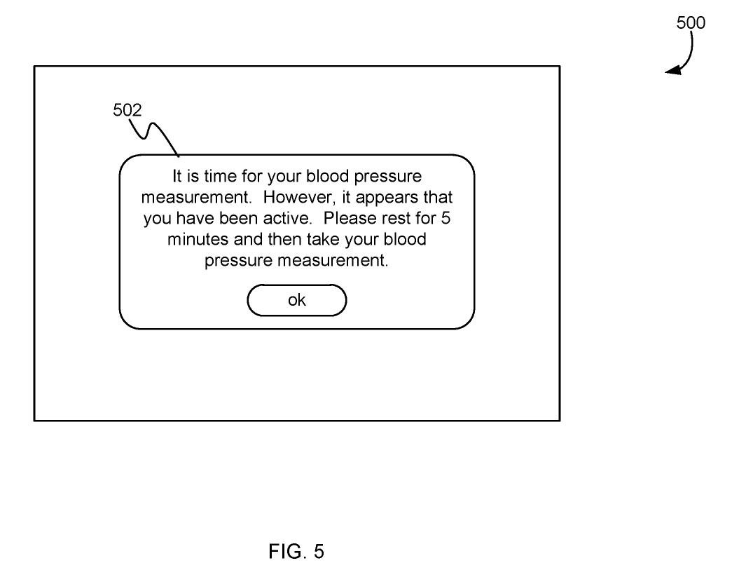

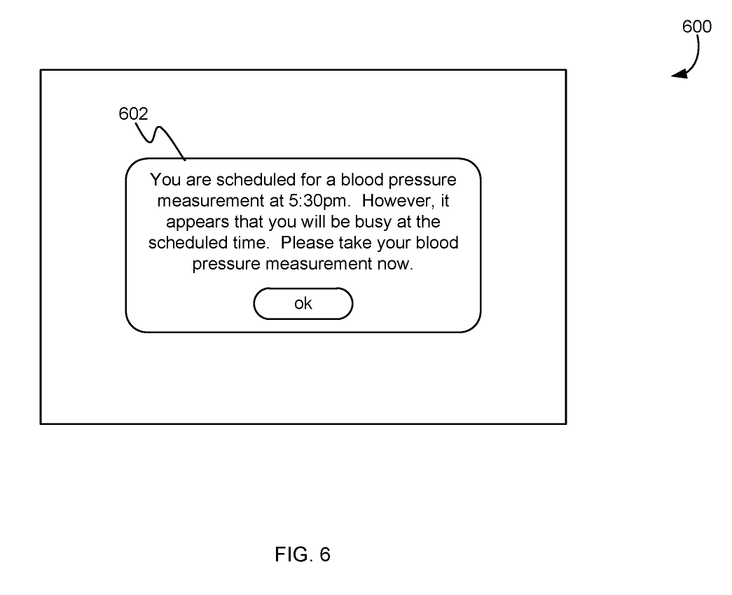

In some implementations, blood pressure monitor 112 can present a reminder notification on user device 110 based on a user's context at or near the time when a reminder is scheduled. For example, rather than just presenting a reminder at a specified time, blood pressure monitor 112 can determine whether the user's context at or near a scheduled time is appropriate for the blood pressure measurement and present a notification (e.g., notification 502 of FIG. 5, notification 602 of FIG. 6) suggesting a modification to the prescribed time and/or user context so that the blood pressure measurement does not produce erroneous results.

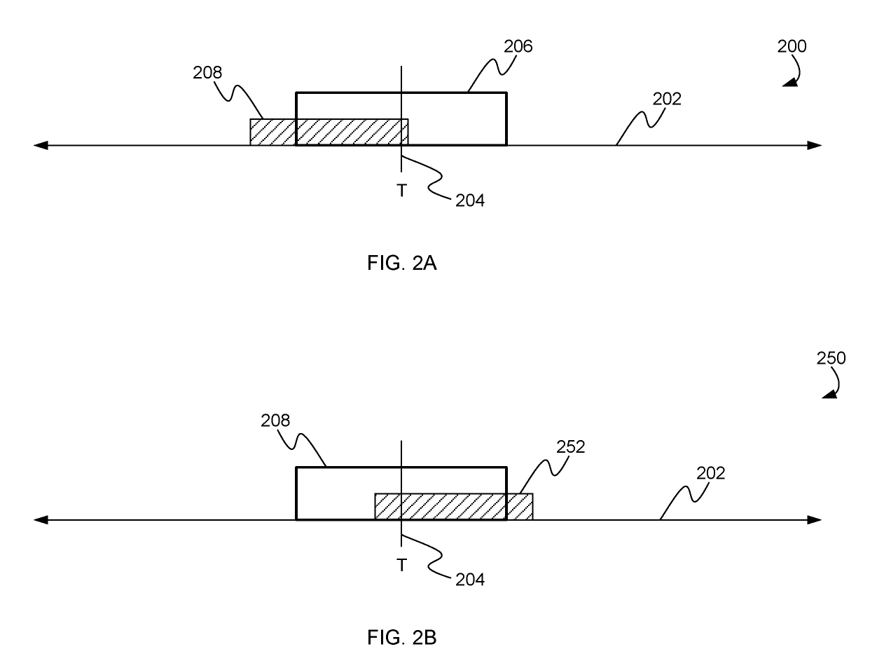

FIG. 2A and FIG. 2B are example graphs 200 and 250 illustrating adapting a blood pressure measurement schedule based on user context. For example, graph 200 of FIG. 2A illustrates adapting a blood pressure measurement schedule based on past or current user context that might generate an erroneous blood pressure measurement. Graph 250 of FIG. 2B illustrates adapting a blood pressure measurement schedule based on a current or future user context that might generate an erroneous blood pressure measurement. For example, if the user is attempting to take a resting blood pressure measurement at a time when the user is excited or stressed, the blood pressure measurement may not be an accurate representation of the user's resting blood pressure. Thus, blood pressure monitor 112 can suggest a time best suited for measuring the user's resting blood pressure.

Graph 200 of FIG. 2A includes a timeline 202 during which a blood pressure measurement is scheduled at time ‘T’ 204. In a basic implementation, blood pressure monitor 112 could simply present a notification reminding the user to measure the user's blood pressure at time ‘T’. However, in some implementations, blood pressure monitor 112 can adjust the reminder notification and/or suggest an alternate time for measuring blood pressure based on the user's context during a window of time 206 that includes time ‘T’. For example, the user may not need to take a blood pressure measurement exactly at time ‘T’; it may be sufficient to measure the user's blood pressure within a 10 minute window (e.g., T-5 minutes to T+5 minutes). By adding some flexibility to the blood pressure measurement schedule, blood pressure monitor 112 can adjust the reminder notifications and/or suggest a different time for measuring the user's blood pressure within window 206 based on the user's context.

Graph 200 includes user stress period 208. For example, user stress period 208 can be a period of time during which the user is experiencing some physical stress and/or psychological stress that might influence a blood pressure measurement and produce erroneous results.

In some implementations, blood pressure monitor 112 can determine user stress period 208 when user device 110 detects a user context that corresponds to physical stress. For example, user device 110 can detect physical stress based on detected motion corresponding to activities such as running, walking, lifting heavy objects, climbing stairs, etc. User device 110 can detect the user's motion using a motion sensor (e.g., accelerometer, gyroscope, etc.). User device 110 can identify the user's activities based on a pattern of motion data generated by the motion sensor. On the other hand, user device 110 can detect or infer the absence of physical stress when user device 110 does not detect motion greater than a threshold amount (e.g., magnitude, frequency, etc.) of motion.

In some implementations, blood pressure monitor 112 can determine user stress period 208 when user device 110 detects a user context that corresponds to psychological stress. Psychological stressors can include environmental conditions (e.g., loud noises, traffic, etc.), interactions with other people (e.g., telephone calls, meetings, etc.), locations (e.g., office, doctor's office, courtroom, etc.), etc. Psychological stress can be detected by blood pressure monitor 112 based on sensor data, application usage, and/or other user input to user device 110 associated with psychological stress. For example, user device 110 can include a microphone (e.g., sound sensor) that can detect loud noises that might increase the stress level of the user. User device 110 can include motion sensors and/or navigation systems that can detect when user device 110 is moving at a speed that correlates to the user driving (e.g., a stressful activity). User device 110 can include an application or function for making telephone calls that might increase the stress level of the user. User device 110 can include a calendar application that includes information describing scheduled meetings that might influence the user's stress level. User device 110 can include navigation application and/or subsystem that can determine the user's location. Blood pressure monitor 112 can compare the user's location with map data to determine whether the user is at a stressful location (e.g., work, doctor's office, etc.). Blood pressure monitor 112 can determine or infer whether the user is influenced by psychological stressors when user device 110 detects any of the above psychological stressors. An absence of psychological stress can be determined or inferred by blood pressure monitor 112 when user device 110 does not detect any of the psychological stressors described above.

If user stress period 208 occurs within a period of time before or during the scheduled blood pressure measurement at time ‘T’, a blood pressure measurement taken at time ‘T’ may be high and not representative of the user's resting blood pressure. To avoid an erroneous measurement in this situation, blood pressure monitor 112 can delay the blood pressure measurement reminder notification or suggest that the user wait to measure the user's blood pressure until a time after time ‘T’ but within window 206 to give the user a chance to relax and reach a resting state. For example, blood pressure monitor 112 can present notification 502 on graphical user interface 500 of FIG. 5 to remind the user that the user should take a blood pressure measurement and to suggest that the user rest for a period of time before taking the blood pressure measurement.

In some implementations, blood pressure monitor 112 can present a reminder notification at time ‘T’ that indicates a blood pressure measurement is due at time ‘T’ and suggest that the user rest for a period of time before taking the blood pressure measurement at some later time within window 206. For example, when blood pressure monitor 112 detects user stress period 208 is occurring within a threshold period of time before time ‘T’, blood pressure monitor 112 can present the reminder notification suggesting that the user rest for a period of time before measuring the user's blood pressure. For example, the rest period can be a period of time determined empirically and configured in blood pressure monitor 112. If no user stress period 208 is detected at or near time ‘T’, blood pressure monitor 112 can present the reminder notification as scheduled at time ‘T’. For example, when blood pressure monitor 112 does not detect user stress period 208 for at least a threshold period of time (e.g., corresponding to the aforementioned rest period) before time ‘T’, blood pressure monitor 112 can present the blood pressure measurement reminder at time ‘T’ as scheduled.

Graph 250 of FIG. 2B is similar to graph 200 of FIG. 2B except that user stress period 252 is predicted to occur during or after time ‘T’. For example, when window 208 begins (e.g., at time T-5 minutes) blood pressure monitor 112 can predict that user stress period 252 will include time ‘T’. For example, blood pressure monitor 112 can predict user stress period 252 based on historical user data indicating that the user may participate in some stressful activity (e.g., either physical or psychological) during a time period corresponding to user stress period 252. Blood pressure monitor 112 can predict user stress period 252 based on application data (e.g., scheduled meetings in a calendar application) that indicates the user may be stressed during user stress period 252. Blood pressure monitor 112 can determine or predict that user stress period 252 is not currently occurring and/or will not occur for a period of time near the beginning of window 208 based on sensor data, application data, user input, etc., as described above. When blood pressure monitor 112 predicts user stress period 252 will occur in during a future period of time that includes time ‘T’ and determines that the user will not be stressed at the beginning of window 208, blood pressure monitor 112 can adjust the blood pressure measurement reminder scheduled for time ‘T’ so that the reminder notification is presented before the scheduled time ‘T’. For example, blood pressure monitor 112 can present the blood pressure measurement reminder scheduled for time ‘T’ before the predicted start time of user stress period 252 and within window 208 to avoid an erroneous measurement at time ‘T’ during a stressful event. For example, blood pressure monitor 112 can present GUI 600 of FIG. 6, including a notification 602 for prompting the user to take a blood pressure measurement in advance of time ‘T’, on a display of user device 110 when blood pressure monitor 112 has determined that the user stress period 252 will include the scheduled time ‘T’.

Referring back to FIG. 1, in some implementations, blood pressure monitor 112 can receive blood pressure measurements. For example, blood pressure monitor 112 can present graphical user interface 300 of FIG. 3 for receiving blood pressure measurement input from a user. Blood pressure monitor 112 can present GUI 300 on a display of user device 110, for example. When blood pressure monitor 112 determines that the user should take a blood pressure measurement, blood pressure monitor 112 can present notification 302 prompting the user to take a blood pressure measurement. The user can measure the user's blood pressure using blood pressure measurement device 130, for example. Blood pressure measurement device 130 can be a standard blood pressure measurement device (e.g., an inflatable cuff and/or pressure sensor). Blood pressure measurement device 130 can be used by the user to measure the user's heart rate as well. After measuring the user's blood pressure and/or heart rate using blood pressure measurement device 130, the user can input the blood pressure measurements and/or heart rate measurements into graphical element 304 (e.g., systolic measurement), graphical element 306 (e.g., diastolic measurement), and graphical element 307 (e.g., heart rate) and submit the measurements to blood pressure monitor 112 by selecting graphical element 308.

In some implementations, blood pressure measurement device 130 can be a networked device that can transmit blood pressure measurements to blood pressure monitor 112 on user device 110 through network 150. For example, the user can initiate a blood pressure measurement at blood pressure measurement device 130. After taking the blood pressure measurement, blood pressure measurement device 130 can transmit the blood pressure measurement (e.g., systolic pressure, diastolic pressure, heart rate, etc.) to blood pressure monitor 112 on user device 110.

Alternatively, the user of user device 110 can initiate a blood pressure measurement using graphical user interface 400 of FIG. 4. In some implementations, blood pressure monitor 112 can present GUI 400 on a display of user device 110 in response to determining that a blood pressure measurement reminder should be presented to the user (e.g., according to the prescribed schedule) and/or when blood pressure monitor 112 is connected to blood pressure measurement device 130. Blood pressure monitor 112 can present notification 402 when user device 110 is connected or was previously connected (e.g., paired, configured to connect, etc.) to blood pressure monitor 112. The user can prepare blood pressure measurement device 130 (e.g., an inflatable cuff) for taking the measurement by putting blood pressure measurement device 130 on the user's arm. After blood pressure measurement device 130 is in place, the user can select graphical element 404 (e.g., a start button) to cause blood pressure measurement device 130 to take the blood pressure measurement. For example, user device 110 can send a message to blood pressure measurement device 130 to initiate the blood pressure measurement. After blood pressure measurement device 130 measures the user's blood pressure and/or heart rate, blood pressure measurement device 130 can send the measurements (e.g., systolic pressure, diastolic pressure, heart rate, etc.) to user device 110 through network 150.

In some implementations, blood pressure monitor 112 can store received blood pressure measurements in blood pressure database 116. For example, when blood pressure monitor 112 receives a blood pressure measurement (e.g., by user input, by message transmitted through network 150 from blood pressure measurement device 130), blood pressure monitor 112 can store the blood pressure measurements and/or heart rate in blood pressure database 116. In some implementations, blood pressure monitor 112 can store user context information in association with blood pressure measurements. For example, blood pressure monitor 112 can store data describing physical and/or psychological stressors detected by user device 110 at or about the time when the user measured or entered the user's blood pressure, as described above.

In some implementations, system 100 can include multiple user devices that can be configured to cooperate to intelligently monitor blood pressure. For example, system 100 can include wearable device 120. Wearable device 120 can be a smart watch, smart glasses, smart clothing, or other wearable device. Wearable device 120 can include some or all of the features of user device 110 described above and below.

In some implementations, wearable device 120 can include blood pressure monitor 122. For example, blood pressure monitor 122 can be configured with the same or similar functionality as blood pressure monitor 112 of user device 110, described above. For example, blood pressure monitor 122 can schedule blood pressure measurement reminders and/or notifications according to a prescribed schedule and/or user context, as described above.

In some implementations, blood pressure monitor 122 can be configured to share device data with user device 110. For example, wearable device 120 and user device 110 can be owned or used by the same user and configured to share device data between the devices. Wearable device 120 and user device 110 can share or synchronize device data through network 150, for example. The device data can include sensor data (e.g., movement, light, noise sensor data), application usage information (e.g., describing the applications currently used by the user on each device), and/or blood pressure measurement data (e.g., measurements taken, adjustments to measurement schedules, etc.). Thus, blood pressure monitor 122 of wearable device 120 and blood pressure monitor 112 of user device 110 can synchronize data and operations so that actions, measurements, schedules, and/or schedule adjustments made on one device will be reflected in the measurement data, schedule, and operations of the other device.

In some implementations, blood pressure monitor 122 on wearable device 120 can automatically take blood pressure measurements. For example, wearable device 120 can be configured with blood pressure sensor 124 and/or heart rate sensor 126. Thus, in some implementations, instead of reminding the user to take a blood pressure measurement at a scheduled time or adjusted time, blood pressure monitor 122 can automatically take the blood pressure measurement at the scheduled or adjusted time (as described above) using blood pressure sensor 124 on wearable device 120. In some implementations, blood pressure monitor 122 can take a heart rate measurement at or near the time of a blood pressure measurement using heart rate sensor 126 of wearable device 120.

In some implementations, blood pressure monitor 122 can automatically measure the user's blood pressure in response to detecting different user contexts. For example, instead of, or in addition to, determining a suitable user context for measuring a resting blood pressure, as described above, blood pressure monitor 122 can determine the appropriate user contexts for measuring a resting blood pressure, an active blood pressure, and a high stress blood pressure. Resting blood pressure can, for example, be measured when blood pressure monitor 122 detects that the user is resting (e.g., not experiencing physical and/or psychological stress), as described above. High stress blood pressure (e.g., stress test) can be measured when blood pressure monitor 122 detects that the user is experiencing or participating in a high stress activity, such as running, a business meeting, etc. Active blood pressure can be measured when blood pressure monitor 122 detects that the user is neither resting nor participating in a high stress activity. In some implementations, blood pressure monitor 122 can be configured to automatically measure the user's blood pressure on a periodic basis corresponding to a configured time interval. After blood pressure monitor 122 measures the user's blood pressure, blood pressure monitor 122 can store the blood pressure measurement and/or user context information in blood pressure database 126.

In some implementations, blood pressure monitor 122 can determine whether the user is experiencing psychological stress based on physiological measurements. For example, wearable device 120 can include a heart rate monitor 120 that can measure the user's heart rate while the user is wearing device 120. For example, a high heart rate in the absence of physical activity can be an indicator of psychological stress. Blood pressure monitor 122 can monitor the user's heart rate and prompt the user to take a blood pressure measurement when the user's heart rate is high (e.g., above a high threshold value) or low (e.g., below a low threshold value). When wearable device 120 is configured with blood pressure sensor 124, blood pressure monitor 122 can automatically measure the user's blood pressure in response to detecting a high or low heart rate condition.

In some implementations, blood pressure monitor 122 can initiate a blood pressure measurement in response to user input. For example, the user of wearable device 120 may feel faint and initiate a blood pressure measurement by providing input to wearable device 120. In response to the user input, blood pressure monitor 122 can measure the user's blood pressure using blood pressure sensor 124 and/or the user's heart rate using heart rate sensor 126. In some implementations, blood pressure monitor 122 can collect contextual data in response to the user initiating a blood pressure measurement. For example, blood pressure monitor 124 can use a sound sensor (e.g., microphone) to detect the ambient noise level, location sensor to detect the user's current location, and/or other sensors to detect other user context information, as described above. For example, blood pressure monitor 124 can use a motion sensor (e.g., accelerometer) to detect the user's movements and/or posture at the time the blood pressure measurement was taken. The user's posture can be useful, for example, for a doctor to diagnose a postural hypotension or other postural blood pressure anomaly. This contextual data can be stored in correlation with the blood pressure measurements (e.g., in blood pressure database 116 and/or 126) to aid a physician or user in diagnosing blood pressure issues.

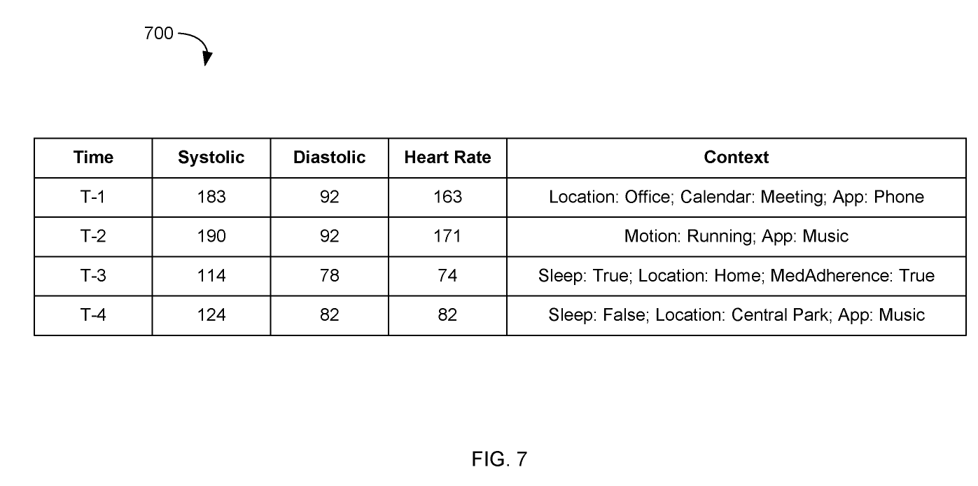

FIG. 7 illustrates an example blood pressure measurement database 700. For example, blood pressure database 700 can correspond to blood pressure database 116 and/or blood pressure database 126 of FIG. 1. In some implementations, blood pressure database 700 can include records (e.g., rows) corresponding to each blood pressure measurement received by blood pressure monitor 112. For example, each blood pressure record can include the time at which the user's blood pressure was measured, the blood pressure measurements (e.g., systolic and/or diastolic pressure), the user's heart rate, and/or the user context at the time when the user's blood pressure was measured. For example, the user context can include the user's location, calendar events, application usage, motion data, sleep state, medication adherence information (e.g., if a medication adherence application is being used to track medication adherence), ambient noise level, or a combination thereof. By storing blood pressure measurements and user context information, user device 110 and/or wearable device 120 can provide to the user both blood pressure measurements and context information that may explain why a blood pressure reading is particularly high or low. The user can use the context information and blood pressure readings to get a better sense of what a normal blood pressure measurement is for the user.

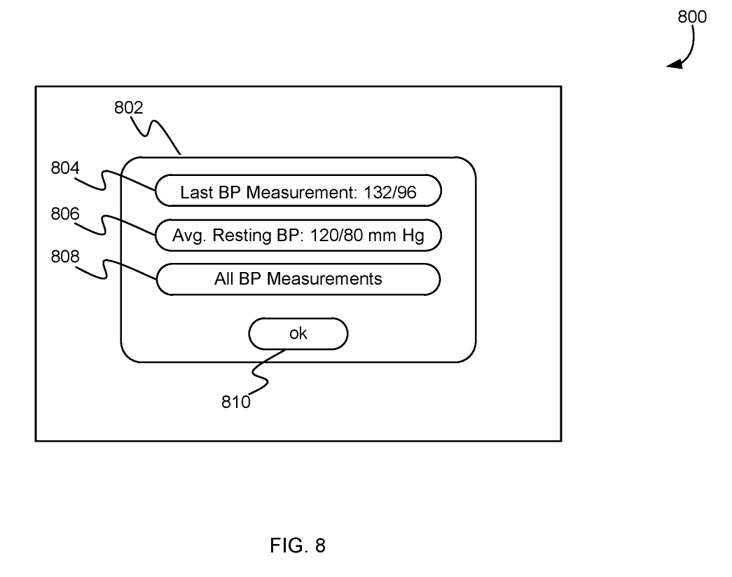

FIG. 8 illustrates an example graphical user interface 800 for presenting a blood pressure measurement summary to the user. For example, GUI 800 can be presented on a display of user device 110 and/or wearable device 120 in response to receiving user input indicating that the user would like to view a blood pressure summary. Alternatively, GUI 800 can be presented automatically at some user-configured time or periodically according to some user-configured time interval.

In some implementations, GUI 800 can include blood pressure summary notification 802. For example, notification 802 can include graphical element 804 describing the last blood pressure measurement. The user can select graphical element 804, for example, to view context information that may explain the blood pressure measurement. Notification 802 can include graphical element 806 describing the user's average resting blood pressure. The user can select graphical element 806 to view details about the average resting blood pressure calculation. For example, user device 110 and/or wearable device 120 can present a chart that describes individual resting blood pressure measurements that were used to calculate the average resting blood pressure. Notification 802 can include graphical element 808 for viewing a chart that describes all blood pressure measurements (e.g., taken over a preceding period of time). The user can dismiss notification 802 by selecting graphical element 810.

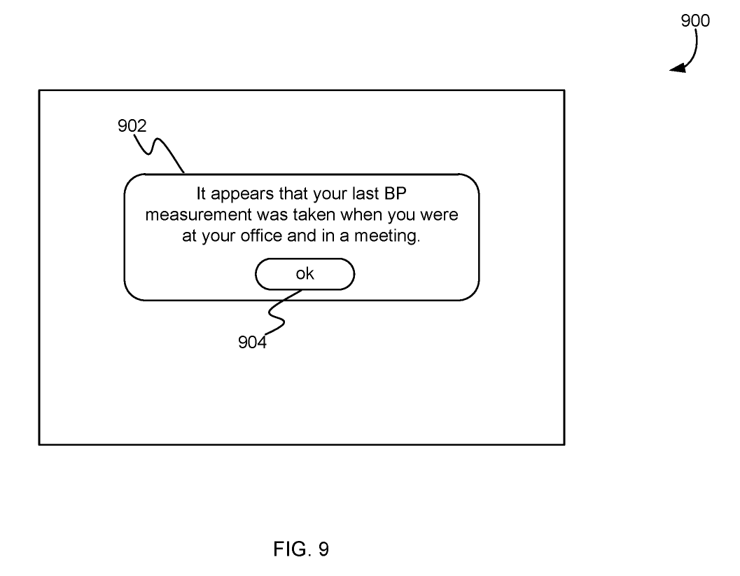

FIG. 9 illustrates an example graphical user interface 900 for describing the user context associated with a blood pressure measurement. For example, GUI 900 can be presented on a display of user device 110 by blood pressure monitor 112 in response to a user selection of graphical element 802 describing the last blood pressure measurement recorded by blood pressure monitor 122. For example, GUI 900 can include notification 902 that includes text describing the user context (e.g., sensor readings, application usage, location, user activity, etc.) at the time the blood pressure measurement was taken or recorded. Thus, if the last blood pressure measurement appears to the user to be a high measurement, the user can determine based on the information presented on notification 902 whether the high blood pressure measurement was appropriate for the user context at the time of the reading or whether the user should consult a physician to determine if the user has any health issues that might be causing an abnormally high blood pressure measurement. The user can select graphical element 904 to dismiss notification 902.

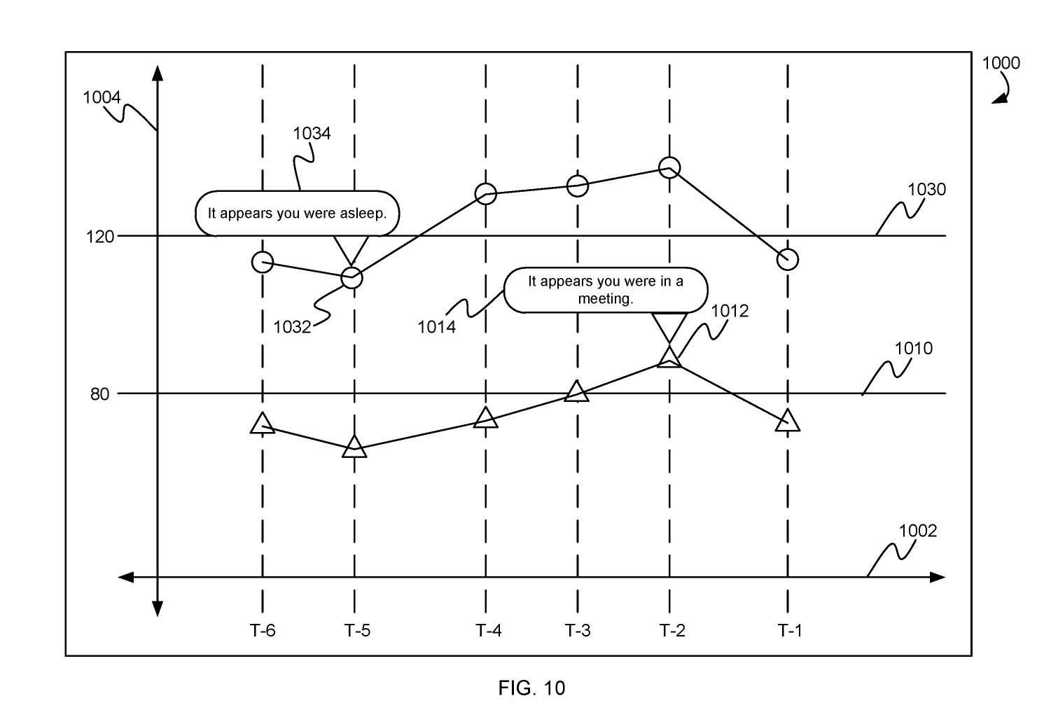

FIG. 10 illustrates an example graphical user interface 1000 for presenting a detailed view of a user's recorded blood pressure measurements. For example, GUI 1000 can be presented by blood pressure monitor 112 in response to the user selecting graphical element 806 or graphical element 808. GUI 1000 can present a graph that shows blood pressure measurements that have been recorded in blood pressure database 116, for example. When GUI 1000 is presented in response to a user selection of graphical element 806, GUI 1000 can present resting blood pressure measurements. For example, blood pressure monitor 112 can filter the blood pressure measurements recorded in blood pressure database 116 based on user context so that only resting blood pressure measurements are presented on GUI 1000.

When GUI 1000 is presented in response to a user selection of graphical element 808, blood pressure monitor 112 can present unfiltered blood pressure measurements recorded in blood pressure database 116. For example, blood pressure monitor 112 can present a previous number (e.g., 10, 15, 23, etc.) of measurements. The blood pressure measurements can include resting, active, and/or stressed blood pressure measurements, for example. Blood pressure monitor 112 can present blood pressure measurements taken during a previous time period (e.g., the last 1 day, 2 days, 1 week, etc.). The number of blood pressure measurements and/or the time period can be user-configurable values, for example.

In some implementations, GUI 1000 can present a graph of blood pressure measurements. For example, GUI 1000 can include timeline axis 1002 corresponding to a period of time for which blood pressure measurements will be presented. GUI 1000 can include measurement axis 1004 corresponding to a range of blood pressure measurements (e.g., in mmHg) stored in blood pressure database 116. In some implementations, GUI 1000 can include blood pressure reference lines 1010 (e.g., diastolic) and 1030 (e.g., systolic) corresponding to normal diastolic and systolic blood pressure measurements. GUI 1000 can include systolic (circles) and diastolic (triangle) measurements at corresponding times (e.g., T-6, T-5, etc.) along timeline 1002. By comparing the blood pressure measurements to the reference lines 1010 and/or 1030, the user can quickly determine how the user's blood pressure measurements compare to normal blood pressure measurements.

In some implementations, GUI 1000 can present user context information for blood pressure measurements presented on GUI 1000. For example, a user can select a blood pressure measurement (e.g., systolic measurement 1032) to cause GUI 1000 to present graphical element 1034 that includes text, graphics, or other features describing the recorded user context at the time (e.g., T-5) of the selected measurement. Similarly, a user can select diastolic blood pressure measurement 1012 to cause GUI 1000 to present graphical element 1014 describing the recorded user context at the time (e.g., T-2) when blood pressure measurement 1012 was recorded or measured.

Referring back to FIG. 1, in some implementations, user device 110 can transmit blood pressure measurements to doctor device 140. For example, when the doctor associated with doctor device 140 writes a prescription for blood pressure measurements, the prescription can be transmitted to user device 110 through network 150, as described above. When user device 110 receives the prescription, user device 110 can present a prompt (e.g., a notification, graphical element, etc.) on a display of user device 110 asking the user to provide input to accept the prescription. In some implementations, the prompt can also ask the user if the user wishes to automatically send blood pressure measurements to the doctor (e.g., doctor device 140). When the user provides input indicating that the user wishes to automatically send blood pressure measurements to doctor device 140, user device 110 can automatically send blood pressure measurements to doctor device 140. For example, user device 110 can automatically send blood pressure measurements recorded in blood pressure database 116 to doctor device 140 on a periodic basis (e.g., every 24 hours, once a week, etc.) or in response to recording a blood pressure measurement in blood pressure database 116.

### Example Processes

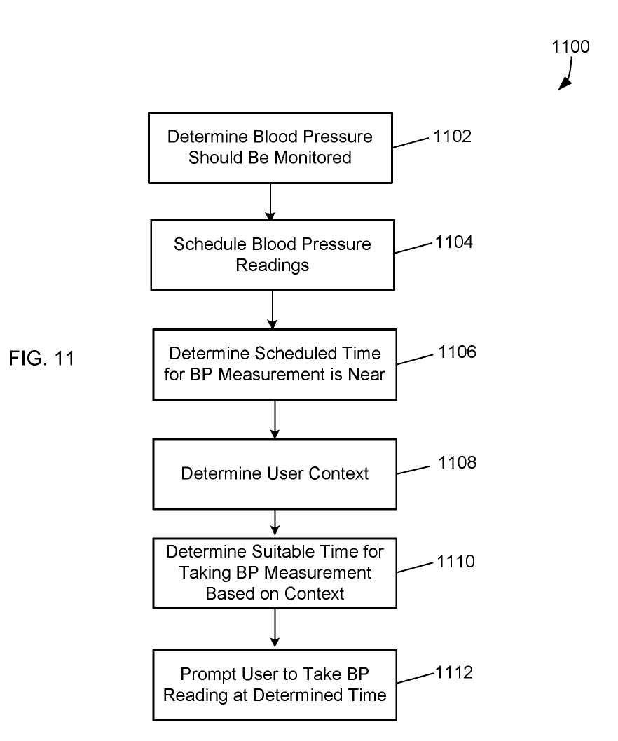

FIG. 11 is flow diagram of an example process 1100 for intelligent blood pressure monitoring. For example, process 1100 can intelligently present blood pressure measurement reminders on a display of a user device based on a prescribed schedule and/or user context. For example, process 1100 can be implemented on the user device so that the user device can determine a suitable time to remind the user to take a resting blood pressure measurement.

At step 1102, a user device can determine that a user's blood pressure should be monitored. For example, the user device can correspond to user device 110 and/or wearable device 120 of FIG. 1. The user device can determine that the user's blood pressure should be monitored when a blood pressure measurement prescription is received by the user device. For example, the prescription can be input manually (e.g., transcribed into a graphical user interface) by a user of the user device. The prescription can be transmitted to the user device over a network connection (e.g., from a doctor's device, pharmacist's device, microchip embedded in a prescription label, etc.). The user device can determine that the user's blood pressure should be monitored when the user turns on a blood pressure monitoring feature of the user device.

At step 1104, the user device can schedule blood pressure readings according to the prescription. For example, the blood pressure monitoring prescription can describe (prescribe) a schedule for measuring and/or recording the user's blood pressure. The user device can schedule blood pressure measurement reminder notifications according to the prescribed schedule.

At step 1106, the user device can determine that the scheduled time for a blood pressure measurement is near. For example, the user device can schedule a window of time during which the user device should remind the user to take a blood pressure measurement, as described above with reference to FIG. 3. For example, the window of time can be the scheduled time ‘T’ plus and minus 5 minutes (e.g., a 10 minute window). The user device can schedule (e.g., using an internal timer or alarm) a user context check for T-5 minutes in advance of the scheduled blood pressure measurement at time ‘T’.

At step 1108, the user device can determine the user context for the scheduled blood pressure measurement. For example, at T-5 minutes the user device can determine the current user context, the predicted user context at time ‘T’, or a predicted future user context after time ‘T’ and within the time window. For example, the user device can determine the current user context based on sensor data, application usage, location information, etc., as described above. The user device can determine the future user context based on historical data (e.g., user behavior patterns), calendar application data indicating future meetings or events, and/or other application data that may indicate what activities in which the user may be involved in the future.

At step 1110, the user device can determine a suitable time for taking the blood pressure measurement based on the user context. For example, the user device can determine based on the user context a time during the scheduled time window when the user is most likely to be resting so that the user can take a resting blood pressure measurement.

At step 1112, the user device can prompt the user to take the blood pressure measurement at the determined time. For example, the user device can present a prompt on a display of the user device suggesting that the user take a blood pressure measurement before time ‘T’, at time ‘T’, or after time ‘T’, as described above with reference to FIGS. 3-6. After the user measures the user's blood pressure (e.g., using a sensor of the user device or an external device), the user device can store the blood pressure measurement and/or context information.

Although the process 1100 describes presenting reminders to prompt the user to take blood pressure measurements according to a prescribed schedule, the user device (e.g., wearable device 120) can be configured to automatically measure the user's blood pressure in lieu of presenting reminders at the determined times, as described above.

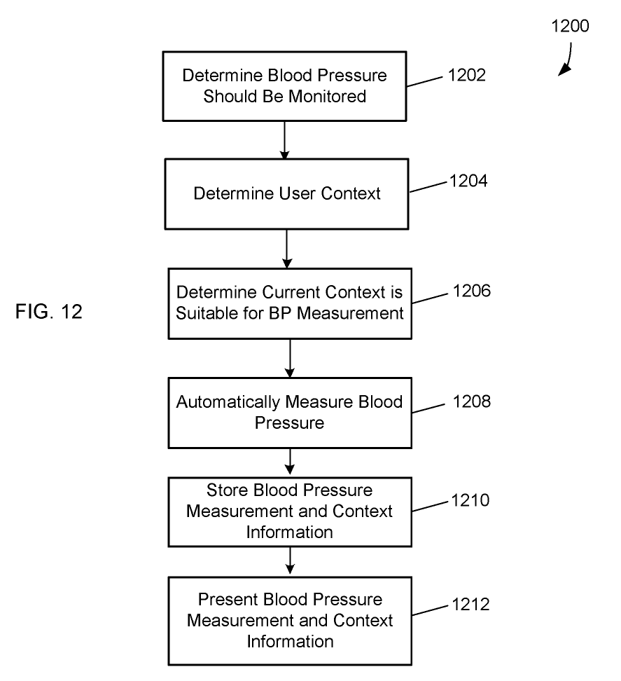

FIG. 12 is a flow diagram of an example process 1200 for automatic blood pressure monitoring. For example, process 1200 can automatically measure a user's blood pressure based on a prescribed schedule and/or user context. For example, process 1100 can be implemented on the user device so that the user device can determine a suitable time to automatically measure the user's resting, active, and stressed blood pressure.

At step 1202, a user device can determine that a user's blood pressure should be monitored. For example, the user device can correspond to user device 110 and/or wearable device 120 of FIG. 1. The user device can determine that the user's blood pressure should be monitored when a blood pressure measurement prescription is received by the user device. For example, the prescription can be input manually (e.g., transcribed into a graphical user interface) by a user of the user device. The prescription can be transmitted to the user device over a network connection (e.g., from a doctor's device, pharmacist's device, microchip embedded in a prescription label, etc.). The prescription can specify a schedule for taking blood pressure measurements. The prescription can specify the types of blood pressure measurements (e.g., resting, active, stressed, etc.) to be collected by the user device. The user device can determine that the user's blood pressure should be monitored when the user turns on a blood pressure monitoring feature of the user device.

At step 1204, the user device can determine the current user context. For example, the user device can determine the current user context based on sensor data, application usage information, location information, etc., collected by the user device and/or other user devices. For example, the user device 110 can receive user context information from wearable device 120 describing the user's movements (e.g., motion sensor data), location, heart rate, application usage, etc.

At step 1206, the user device can determine that the current user context is suitable for a blood pressure measurement. For example, when the user device is configured to measure the user's resting blood pressure, the user device can determine that the current user context is suitable for measuring the user's resting blood pressure when the current user context does not include any physical and/or psychological stressors. When the user device is configured to measure the user's stressed blood pressure, the user device can determine that the current user context is suitable for measuring the user's stressed blood pressure (e.g., for performing a stress test) when the current user context includes physical and/or psychological stressors, such as running or a business meeting. When the user device is configured to measure the user's active blood pressure, the user device can determine that the current user context is suitable for measuring the user's active blood pressure when the current user context is not a resting context and also is not a stressed context, as described above.

At step 1208, the user device can automatically measure the user's blood pressure. For example, when the user device includes a blood pressure sensor (e.g., wearable device 120), the user device can automatically measure the user's blood pressure when the user context is suitable for taking the desired type of blood pressure measurement. For example, in response to determining that the current user context indicates the user is resting, the user device can measure the user's resting blood pressure. In response to determining that the current user context indicates the user is stressed, the user device can measure the user's stressed blood pressure. In response to determining that the current user context indicates the user is participating in normal, everyday activities (e.g., not resting, not stressed), the user device can measure the user's active blood pressure. In some implementations, the blood pressure measurement can be performed as a background task on the user device without notification or prompt to the user.

At step 1210, the user device can store the blood pressure measurement and/or context information. For example, the user device can store the automatically measured blood pressure measurement and/or context information describing the user's context at the time when the user's blood pressure was measured in a database on the user device, as described with reference to FIG. 7 above.

At step 1212, the user device can present the blood pressure measurement and context information stored in the blood pressure database to the user. For example, the user can provide input to the user device invoking a graphical user interface for presenting blood pressure measurements and/or context information, as described above with reference to FIGS. 8-10.

In some implementations, the user device can automatically transmit or send blood pressure measurements and/or user context information to a doctor's device for analysis by the doctor. For example, after blood pressure measurements are stored in the blood pressure database, the user device can periodically send the stored blood pressure measurements to the doctor's device through network 150.

While FIG. 11 and FIG. 12 describe specific a specific sequence of steps for processes 1100 and 1200, respectfully, individual steps of processes 1100 and/or 1200 can be rearranged, reordered, omitted, etc., while remaining within the scope of the present disclosure. Moreover, a person of ordinary skill will understand that the steps of process 1100 can be combined with steps of process 1200 and, similarly, the steps of process 1200 can be combined with the steps of process 1100. For example, step 1210 of FIG. 12 can be included in process 1100 so that process 1100 will store the blood pressure measurements taken or received in process 1100.

### Graphical User Interfaces

This disclosure above describes various Graphical User Interfaces (GUIs) for implementing various features, processes or workflows. These GUIs can be presented on a variety of electronic devices (e.g., user device 110 and/or wearable device 120) including but not limited to laptop computers, desktop computers, computer terminals, television systems, tablet computers, e-book readers and smart phones. One or more of these electronic devices can include a touch-sensitive surface. The touch-sensitive surface can process multiple simultaneous points of input, including processing data related to the pressure, degree or position of each point of input. Such processing can facilitate gestures with multiple fingers, including pinching and swiping.

When the disclosure refers to “select” or “selecting” user interface elements in a GUI, these terms are understood to include clicking or “hovering” with a mouse or other input device over a user interface element, or touching, tapping or gesturing with one or more fingers or stylus on a user interface element. User interface elements can be virtual buttons, menus, selectors, switches, sliders, scrubbers, knobs, thumbnails, links, icons, radio buttons, checkboxes and any other mechanism for receiving input from, or providing feedback to a user.

### Privacy

The present disclosure recognizes that the use of personal information data, in the present technology, can be used to the benefit of users. For example, the personal information data can be used to deliver targeted content (e.g., reminders, notifications, etc.) that is of greater interest to the user or more closely tailored to the user's needs. Accordingly, use of such personal information data enables calculated control of the delivered content. Further, other uses for personal information data that benefit the user are also contemplated by the present disclosure.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services. In another example, users can select not to provide location information for targeted content delivery services. In yet another example, users can select to not provide precise location information, but permit the transfer of location zone information.

### Example System Architecture

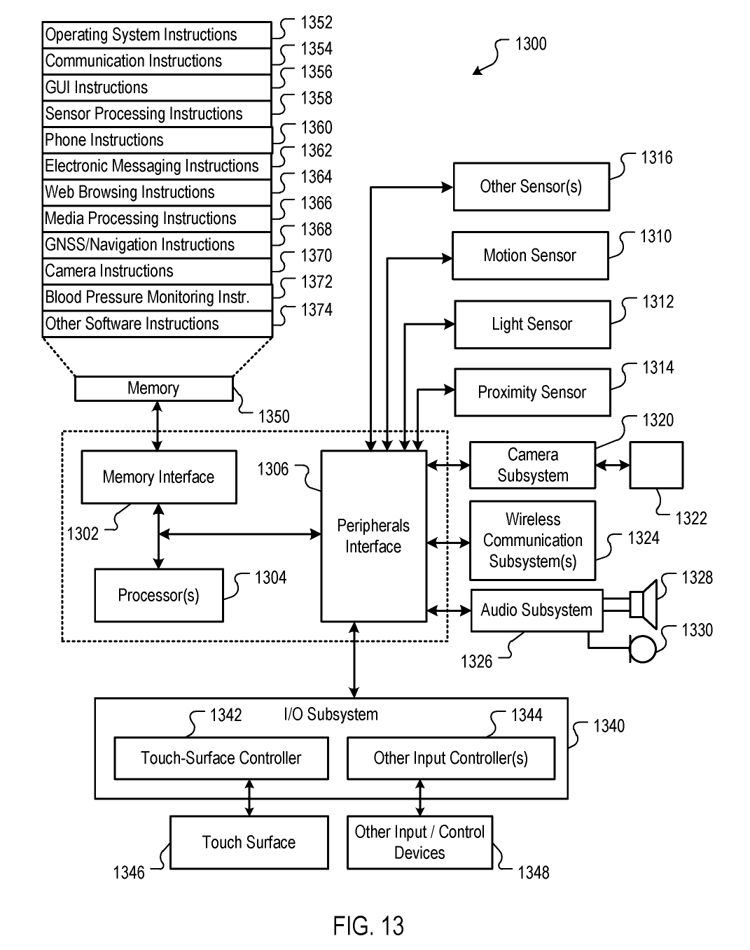

FIG. 13 is a block diagram of an example computing device 1300 that can implement the features and processes of FIGS. 1-12. The computing device 1300 can include a memory interface 1302, one or more data processors, image processors and/or central processing units 1304, and a peripherals interface 1306. The memory interface 1302, the one or more processors 1304 and/or the peripherals interface 1306 can be separate components or can be integrated in one or more integrated circuits. The various components in the computing device 1300 can be coupled by one or more communication buses or signal lines.

Sensors, devices, and subsystems can be coupled to the peripherals interface 1306 to facilitate multiple functionalities. For example, a motion sensor 1310, a light sensor 1312, and a proximity sensor 1314 can be coupled to the peripherals interface 1306 to facilitate orientation, lighting, and proximity functions. Other sensors 1316 can also be connected to the peripherals interface 1306, such as a global navigation satellite system (GNSS) (e.g., GPS receiver), a temperature sensor, a biometric sensor, magnetometer or other sensing device, to facilitate related functionalities.

A camera subsystem 1320 and an optical sensor 1322, e.g., a charged coupled device (CCD) or a complementary metal-oxide semiconductor (CMOS) optical sensor, can be utilized to facilitate camera functions, such as recording photographs and video clips. The camera subsystem 1320 and the optical sensor 1322 can be used to collect images of a user to be used during authentication of a user, e.g., by performing facial recognition analysis.

Communication functions can be facilitated through one or more wireless communication subsystems 1324, which can include radio frequency receivers and transmitters and/or optical (e.g., infrared) receivers and transmitters. The specific design and implementation of the communication subsystem 1324 can depend on the communication network(s) over which the computing device 1300 is intended to operate. For example, the computing device 1300 can include communication subsystems 1324 designed to operate over a GSM network, a GPRS network, an EDGE network, a Wi-Fi or WiMax network, and a Bluetooth™ network. In particular, the wireless communication subsystems 1324 can include hosting protocols such that the device 100 can be configured as a base station for other wireless devices.

An audio subsystem 1326 can be coupled to a speaker 1328 and a microphone 1330 to facilitate voice-enabled functions, such as speaker recognition, voice replication, digital recording, and telephony functions. The audio subsystem 1326 can be configured to facilitate processing voice commands, voiceprinting and voice authentication, for example.

The I/O subsystem 1340 can include a touch-surface controller 1342 and/or other input controller(s) 1344. The touch-surface controller 1342 can be coupled to a touch surface 1346. The touch surface 1346 and touch-surface controller 1342 can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with the touch surface 1346.

The other input controller(s) 1344 can be coupled to other input/control devices 1348, such as one or more buttons, rocker switches, thumb-wheel, infrared port, USB port, and/or a pointer device such as a stylus. The one or more buttons (not shown) can include an up/down button for volume control of the speaker 1328 and/or the microphone 1330.

In one implementation, a pressing of the button for a first duration can disengage a lock of the touch surface 1346; and a pressing of the button for a second duration that is longer than the first duration can turn power to the computing device 1300 on or off. Pressing the button for a third duration can activate a voice control, or voice command, module that enables the user to speak commands into the microphone 1330 to cause the device to execute the spoken command. The user can customize a functionality of one or more of the buttons. The touch surface 1346 can, for example, also be used to implement virtual or soft buttons and/or a keyboard.

In some implementations, the computing device 1300 can present recorded audio and/or video files, such as MP3, AAC, and MPEG files. In some implementations, the computing device 1300 can include the functionality of an MP3 player, such as an iPod™. The computing device 1300 can, therefore, include a 36-pin connector that is compatible with the iPod. Other input/output and control devices can also be used.

The memory interface 1302 can be coupled to memory 1350. The memory 1350 can include high-speed random access memory and/or non-volatile memory, such as one or more magnetic disk storage devices, one or more optical storage devices, and/or flash memory (e.g., NAND, NOR). The memory 1350 can store an operating system 1352, such as Darwin, RTXC, LINUX, UNIX, OS X, WINDOWS, or an embedded operating system such as VxWorks.

The operating system 1352 can include instructions for handling basic system services and for performing hardware dependent tasks. In some implementations, the operating system 1352 can be a kernel (e.g., UNIX kernel). In some implementations, the operating system 1352 can include instructions for performing voice authentication. For example, operating system 1352 can implement the intelligent blood pressure monitoring features as described with reference to FIGS. 1-12.

The memory 1350 can also store communication instructions 1354 to facilitate communicating with one or more additional devices, one or more computers and/or one or more servers. The memory 1350 can include graphical user interface instructions 1356 to facilitate graphic user interface processing; sensor processing instructions 1358 to facilitate sensor-related processing and functions; phone instructions 1360 to facilitate phone-related processes and functions; electronic messaging instructions 1362 to facilitate electronic-messaging related processes and functions; web browsing instructions 1364 to facilitate web browsing-related processes and functions; media processing instructions 1366 to facilitate media processing-related processes and functions; GNSS/Navigation instructions 1368 to facilitate GNSS and navigation-related processes and instructions; and/or camera instructions 1370 to facilitate camera-related processes and functions.

The memory 1350 can store other software instructions 1372 to facilitate other processes and functions, such as the intelligent blood pressure monitoring processes and functions as described with reference to FIGS. 1-12.

The memory 1350 can also store other software instructions 1374, such as web video instructions to facilitate web video-related processes and functions; and/or web shopping instructions to facilitate web shopping-related processes and functions. In some implementations, the media processing instructions 1366 are divided into audio processing instructions and video processing instructions to facilitate audio processing-related processes and functions and video processing-related processes and functions, respectively.

Each of the above identified instructions and applications can correspond to a set of instructions for performing one or more functions described above. These instructions need not be implemented as separate software programs, procedures, or modules. The memory 1350 can include additional instructions or fewer instructions. Furthermore, various functions of the computing device 1300 can be implemented in hardware and/or in software, including in one or more signal processing and/or application specific integrated circuits.

What is claimed is:

1. A method comprising:
determining, by a computing device, that a user of the computing device should measure the user's blood pressure;
scheduling, by the computing device, a time for presenting a notification for reminding the user to measure the user's blood pressure according to a prescribed schedule;
obtaining, by the computing device, user context information describing a context of the user near the scheduled time;
adjusting, by the computing device, the scheduled time for the blood pressure measurement notification based on the user context information; and
presenting, by the computing device, a prompt reminding the user to measure the user's blood pressure at the adjusted time.

2. The method of claim 1, wherein the computing device determines that the user should measure the user's blood pressure in response to receiving a prescription from a second computing device.

3. The method of claim 1, wherein the user context information includes information describing the user's physical activity.

4. The method of claim 1, wherein the user context information includes geographical location information indicating a current or future geographical location of the user.

5. The method of claim 1, wherein the user context information includes application usage information describing a current use of an application on the computing device.

6. The method of claim 1, wherein the user context information includes device data from a third computing device associated with the user.

7. The method of claim 1, wherein the prompt suggests that the user measure the user's blood pressure prior to the scheduled time.

8. The method of claim 1, wherein the prompt suggests that the user measure the user's blood pressure after the scheduled time.

9. The method of claim 1, wherein the prompt suggests that the user rests for a period of time based on a current user context.

10. A non-transitory computer-readable medium including one or more sequences of instructions that, when executed by one or more processors, causes:
determining, by a computing device, that a user of the computing device should measure the user's blood pressure;
scheduling, by the computing device, a time for measuring the user's blood pressure according to a prescribed schedule;
obtaining, by the computing device, user context information describing a context of the user near the scheduled time;
adjusting, by the computing device, the scheduled time for measuring the user's blood pressure based on the user context information; and
automatically measuring, by the computing device, the user's blood pressure at the adjusted time.

11. The method of claim 10, wherein the computing device determines that the user should measure the user's blood pressure in response to receiving a prescription from a second computing device.

12. The method of claim 10, wherein the user context information includes information describing the user's physical activity.

13. The method of claim 10, wherein the user context information includes geographical location information indicating a current or future geographical location of the user.

14. The method of claim 10, wherein the user context information includes application usage information describing a current use of an application on the computing device.

15. The method of claim 10, wherein the user context information includes device data from a third computing device associated with the user.

16. The method of claim 10, wherein the adjusted time is prior to the scheduled time.

17. The method of claim 10, wherein the adjusted time is after the scheduled time.

18. A system comprising:
one or more processors; and
a non-transitory computer-readable medium including one or more sequences of instructions that, when executed by the one or more processors, causes:
determining, by a computing device, that a user of the computing device should measure the user's blood pressure;
obtaining, by the computing device, user context information describing a context of the user;
determining, by the computing device, a time for measuring the user's blood pressure based on the user context information; and
automatically measuring, by the computing device, the user's blood pressure at the determined time.

19. The method of claim 18, wherein the user context information includes information describing the user's physical activity.

20. The method of claim 18, wherein the user context information includes geographical location information indicating a geographical location of the user.

21. The method of claim 18, wherein the user context information includes application usage information describing a current use of an application on the computing device.

22. The method of claim 18, wherein the user context information includes device data from a third computing device associated with the user.
